(19) United States
(12) Patent Application Publication
(10) Pub. No.: US 2006/0172290 A1
(43) Pub. Date: Aug. 3, 2006

(54) Nucleic acids coding for a protein interacting with a porin channel

(76) Inventors: Ulf Rapp, Wurzburg (DE); Veronique Le Mellay, Wurzburg (DE)

Correspondence Address: MAYER & WILLIAMS PC, 251 NORTH AVENUE WEST, 2ND FLOOR, WESTFIELD, NJ, 07090, US

(21) Appl. No.: 10/469,375

(22) PCT Filed: Feb. 27, 2002
(86) PCT No.: PCT/DE02/00741

§ 371 Date: Jul. 2, 2004

(30) Foreign Application Priority Data
Feb. 27, 2001 (DE) 101 10 350.6
Mar. 23, 2001 (DE) 101 14 682.5

(51) Int. Cl. C12Q 1/68; C07H 21/04; C12P 21/06; C07K 14/195; C12N 15/74

(52) U.S. Cl. 435/6; 435/69.1; 435/252.3; 435/471; 530/350; 536/23.7

(57) ABSTRACT

The invention relates to an isolated nucleic acid coding for at least a partial sequence of a protein kinase of the mitogenic signaling cascade, in particular c-raf, wherein the partial sequence interacts with VDAC or BAX channels in mitochondrial membranes, or with a nucleic acid hybridizing said nucleic acid or homologues or derivatives of said nucleic acid, to proteins or peptides coded for by such a nucleic acid and to a screening method.

### FIELD OF THE INVENTION

The invention relates to nucleic acids coding for a protein interacting with porins, in particular VDAC, BAX channels or bacterial porins, and to the uses of such nucleic acids or proteins or peptides coded for by such a nucleic acid in screening methods or to the preparation of pharmaceutical compositions.

### BACKGROUND OF THE INVENTION

The raf protein in its various isoforms c-raf, B-raf and A-raf plays an important role in the regulation of the proliferation and differentiation of cells. raf is an effector kinase of ras and an important member in the mitogenic cytoplasmic protein kinase (MAPK) signaling cascade (see e.g. Daum, O., et al., Trends Biochem. Sci. 19:474-479 (1994)). raf proto-oncogenes are highly conserved genes coding for serine/threonine specific kinases of the cytoplasm. These kinases have functions in the mitogenic signal transduction. This cascade transfers signals from receptor tyrosine kinases via ras, raf, MEK and ERK to targets in the cytoplasm and basically for the regulation of the proliferation and differentiation of the cells. The role of c-raf-1 in the classic mitogenic MAP kinase cascade is rather well researched. For details, reference is made here as an example only to U. R. Rapp in The Oncogene Handbook, T. Curran et al., Eds, Elsevier Science Publishers, The Netherlands, 1988, pages 115-154. c-raf-1 is practically ubiquitous in the human organism.

With the transduction of an apoptosis signal into a cell, there are changes of the permeability of membranes of the mitochondria. An increase of the permeability of these membranes will lead to a translocation of the apoptotic protein cytochrome C into the cytoplasm, thereby finally proteolytic proteins called caspase being activated. These proteins lead to the apoptosis. The permeability of mitochondrial membranes is determined, among other factors, by the permeability of the mitochondrial porin channels VDAC (voltage-dependent anion channel). The permeability is in addition influenced by a protein family to which Bcl-2, Bcl-X_{L}, BAX and BAK belong. Bcl-2 acts protectively and binds probably to VDAC. BAX is an integral membrane protein and promotes apoptosis. BAX and Bcl-2 can heterodimerize, and an overexpression of BAX overcompensates the protective effect of Bcl-2. For a deeper insight, reference is made as an example only to Zhou, M., et al. J. Biol. Chem. 273:11930-11936 (1998) and Shimizu, M., Nature (Asia) 399:483 (1999).

It is common to the members of the Bcl-2 family that there are four regions with amino acid homology, BH1, BH2, BH3 and BH4. These domains are essential for the dimerization and/or the function of the different members. In particular the presence or absence of BH4 seem to play an important role for the antiapoptotic or proapoptotic effect (see e.g. Wang, H.-G. et al., Cell 87:629-638 (1996)). For instance Bcl-2 comprises BH4, whereas BAX does not comprise BH4.

From the document Wang, H.-G. et al., Cell 87:629-638 (1996), it is known in the art that raf-1 interacts with Bcl-2. According to this document, BH4 is essential for this interaction. An association raf-1/BAX has not been found, rather has been negated. The results are subject to discussion.

Various diseases are accompanied by a disturbance of the natural cell death programming. A lacking function of the natural cell death is for instance associated with cancer or autoimmune diseases. In contrast thereto, an excessive cell death comes with diseases such as AIDS and neurodegenerative diseases. But infections with pathogenic bacteria may also be accompanied by a disturbance of the natural cell death, bacterial porins being translocated into a cellular membrane and triggering apoptosis of the target cells there. For instance from the document The EMBO Journal 18:339-352 (1999) it is known in the art that the porin PorB in its different variants translocates from Neisseria gonorrhoeae into artificial membranes as well as into cellular target membranes, in particular the outer mitochondrial membrane. These considerations generally apply to Gram-negative bacteria. The relationship of natural mitochondrial porins to bacterial porins has by the way been described already for instance for Escherichia coli, Salmonella typhimurium and Pseudomonas aeruginosa in the document Biochimica et Biophysica Acta 686:204-214 (1982).

Technical Object.

The invention is based on the technical object to inhibit an undesired cell death and to provide suitable active ingredients for treating diseases accompanied by an undesired cell death, in particular AIDS, neurodegenerative diseases, or infections accompanied by an undesired cell death.

Finding the Invention is Based On.

Various experiments with regard to membrane permeability of VDAC or BAX channels were performed in synthetic membranes. Herein, it was found that with an incubation of VDAC or BAX with c-raf, the channels remain closed. In contrast thereto, the channels are open with an incubation of VDAC or BAX with an inactive form of the c-raf. The found interaction of raf with VDAC or BAX may be performed directly or indirectly by mediating substances, possibly Bcl-2. It can further be assumed that due to the similarity between on the one hand natural mitochondrial porins, VDAC and/or BAX, and on the other hand bacterial porins, the latter can also be inhibited or closed by interaction with raf.

Basics of the Invention.

The invention relates to a nucleic acid coding for at least a partial sequence of a protein kinase of the mitogenic signaling cascade, the partial sequence interacting with porins, in particular VDAC, BAX channels in mitochondrial membranes or with bacterial porin channels or a nucleic acid hybridizing such a nucleic acid or homologues or derivatives of said nucleic acids.

The term nucleic acid in particular includes DNA, RNA and PNA. Further subsumed to the term are double-stranded nucleic acids as well as single-stranded nucleic acids and thus also nucleic acids being complementary to each other. Silent mutations are also nucleic acids according to the invention. Silent mutations are variants in the sequence which do not lead to a functional difference, referred to the interaction with VDAC or BAX, of the variant compared to the natural non-mutated sequence. Silent mutations may be alleles or artificial mutations. Derivatives are also covered by the invention. Derivatives are non-natural chemical modifications. Nucleic acids hybridizing nucleic acids according to the invention are such ones which hybridize under stringent conditions. Stringency typically occurs in a temperature range of 5° C. to 25° C. below the melting temperature at the hybridization. By stringent conditions is meant a hybridization at at least 95% sequence identity, preferably 98%. With regard to the hybridization method on which these definitions are based, reference is made to the document Sambrook, J. M. et al., A laboratory manual, Cold Spring Harbor Laboratory Press (1989) and Southern, E. M., J. Mol. Biol. 98:503 (1975).

As protein kinases of the mitogenic signaling cascade, in particular A-raf, B-raf or c-raf may be used. The nucleic acid according to the invention may also be a raf partial sequence only, namely the kinase domain CR3 or active partial sequences therefrom as well as silent mutations and/or derivatives herefrom. CR3 is above the amino acid 302 in the range up to 648. Insofar the nucleic acid may for instance be a nucleic acid coding for Δraf(26-302). It seems to be important that the nucleic acid codes for an active form of the kinase or kinase-active part hereof or a homologous protein or peptide.

By means of nucleic acids according to the invention, a search can be performed for substances interacting with VDAC, BAX or bacterial porin channels. On the one hand, in case of an indirect interaction, substances can be searched which bind to that part of raf having before been found as essential for the interaction. On the other hand, such nucleic acids may also serve as a model for substances of the same function having a higher efficiency. Therefore, the invention also relates to a screening method for determining substances modulating VDAC, BAX or bacterial porin channels, comprising the following steps: a) a VDAC, BAX or bacterial porin channel, as an option a defined partial sequence thereof only, is incubated with a sample substance, in particular a partial sequence of raf, b) the solution of step a) is brought into contact with a membrane positioned in an electrolyte, c) the membrane of step b) is subjected at a time t=0 to an electrical potential difference between different sides of the membrane, and beginning with t=0 or at a defined time after t=0, the current caused by the potential difference or the charge transport through the membrane is measured preferably in dependence of the time, d) the value measured in step c) is compared to a value which has been measured under identical conditions, however with an inactive or active form of raf. Hereby, it can be determined, for instance with regard to the inactive form, whether there is any inhibition at all, and for instance with regard to the active form, whether the inhibition is stronger than for active raf.

Preferred is a nucleic acid according to the invention wherein the interaction is a reduction of the permeability of VDAC, BAX or bacterial porin channels.

Preferably, the nucleic acid codes for an active protein or peptide comprising the sequence c-raf-1 (338 to 627) or an active fragment herefrom or is a nucleic acid hybridizing such a nucleic acid or codes for a protein or peptide consisting of the sequence c-raf-1 (338 to 627) or is a nucleic acid hybridizing such a nucleic acid. It may also be a cDNA. Preferably, for the purpose of this invention, it is human raf. For research purposes, it may however also be raf of non-human mammals.

The invention further relates to an isolated recombinant vector comprising a nucleic acid according to the invention or an expression plasmid comprising this nucleic acid. For the purpose of a stable expression, a DNA fragment, for instance gag, coding for a suitable viral protein may also be used herein (fusion gag with for instance c-raf or c-raf fragments). By means of the expression plasmid, a transformant may be formed which in turn can be used for preparing the protein or peptide coded for by the nucleic acid. For this purpose, the transformant is cultivated in a suitable way following conventional methods.

The invention further relates to a protein or peptide coded for by a nucleic acid according to the invention.

Subject matter of the invention is finally in particular the use of a nucleic acid according to the invention or of a protein or peptide coded for thereby for preparing a pharmaceutical composition for treating AIDS or neurodegenerative diseases or for protecting non-transformed cells in a tissue containing cancer cells or bacterial infections. It is made use herein of that the nucleic acid according to the invention or the protein or peptide coded for thereby acts in an antiapoptotic manner and prevents a (premature) cell death with the consequence of a corresponding disease. In the case of AIDS, the T-lymphocytes are protected. In the case of neurodegenerative diseases, nerve cells are protected from a (irreparable) cell death. For a cancer therapy using cell poisons directed to cells proliferating in an uncontrolled manner, normal healthy cells can be protected from the undesired cell death by the action of the cell poison.

The invention finally relates to a screening method for determining substances activating raf and thus being suitable for preparing pharmaceutical compositions for treating the above diseases, said substances preferably being low-molecular (<10,000 da, preferably <5,000 da). As a screening method is insofar also understood a method for verifying the raf activation by a prospective active ingredient. In principle, such a screening method can be directed to substances which directly or indirectly cause in a cell model an increase of the raf concentration. This may for instance be substances regulating the raf expression up. They may however also be substances which in turn inhibit the raf expression or raf itself. In detail, such screening methods may be configured as follows. A screening method for identifying raf activating substances may comprise the following steps: a) target cells are reacted with a prospective active ingredient or a mixture of prospective active ingredients, and the target cells are cultivated, b) after a defined time, the expression and/or concentration of raf protein is measured, and the obtained measured value is compared to a measured value which has been obtained under identical conditions, however without addition of a prospective active ingredient, c) a prospective active ingredient or a mixture of prospective active ingredients are selected such that an increase of the expression and/or concentration of raf protein was found in step b), d) as an option, in the case of the use of a mixture of prospective active ingredients, a deconvolution is performed, for instance by individual measurement of the active ingredients of the mixture. A screening method for identifying raf activating substances may however also comprise the following steps: a) raf inhibitors naturally occurring in target cells, in particular substances binding to the kinase domain of raf or substances regulating the raf expression down, are identified, b) a substance identified in step a) is as an option isolated and/or purified, reacted in a binding assay with a prospective active ingredient or a mixture of prospective active ingredients, and the binding capability of the substance with the prospective active ingredient is examined, c) a prospective active ingredient or a mixture of prospective active ingredients, for which a binding event has been detected in step b), is selected, d) as an option, in the case of the use of a mixture of prospective active ingredients, a deconvolution is performed, for instance by individual measurement of the active ingredients of the mixture, e) as an option, a selected prospective active ingredient or a selected mixture of prospective active ingredients is subjected to a method according to claim 10 for verifying the raf activity. In principle, all methods for determining a raf concentration and/or a binding assay known to the man skilled in the art can be used. In conjunction with the raf determination, it may be suitable if the used target cells are genetically altered such that expressed raf carries a reporter group, which can easily be detected by measurement and does naturally not exist in the target cells.

Additionally, it is remarked that by an inhibition of raf, to which reference is made in the literature, on the other hand an inhibition of the natural cell death can be prevented for cancer cells, i.e. for lack of raf. VDAC and/or BAX channels are not closed, and apoptosis occurs. Insofar the invention also covers a pharmaceutical composition containing at least one inhibitor of raf for treating diseases, for which the natural apoptosis in cells does not take place, as for instance in cancer cells.

The explanations for one claim category of the invention apply in a corresponding manner for other claim categories. The invention finally also relates to healing methods, for instance classically by administration of pharmaceutical preparations, but also gene therapeutically, by means of which one or several substances according to the invention are brought into a target cell or are produced in the target cell and the expression of which is excited or increased.

In the following, the invention is explained in more detail, based on figures and experiments representing embodiments only. There are:

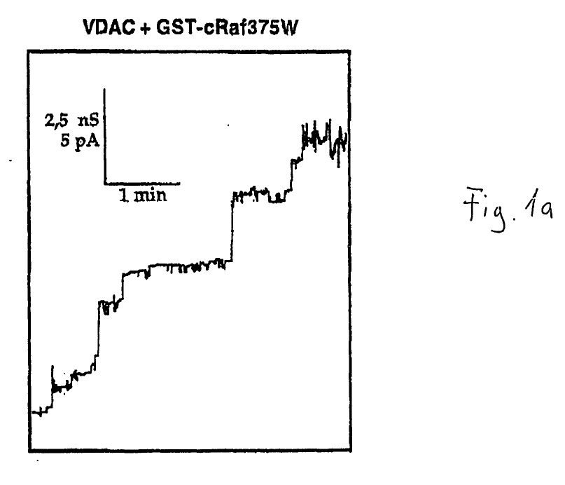

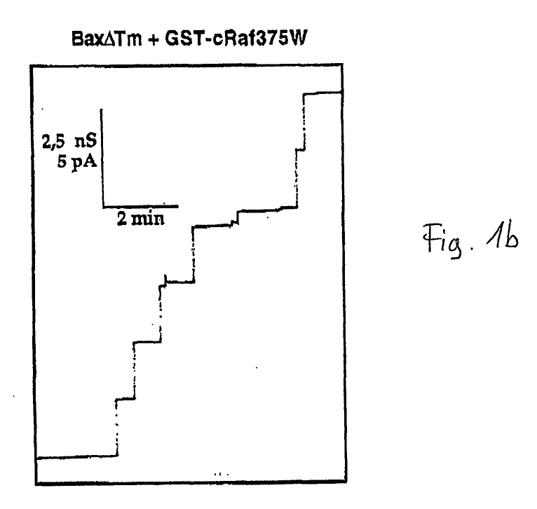

FIG. 1a, b: the permeability of VDAC (a) and BAXΔTM (b) channels after incubation with GST-c-rafL375W (a),

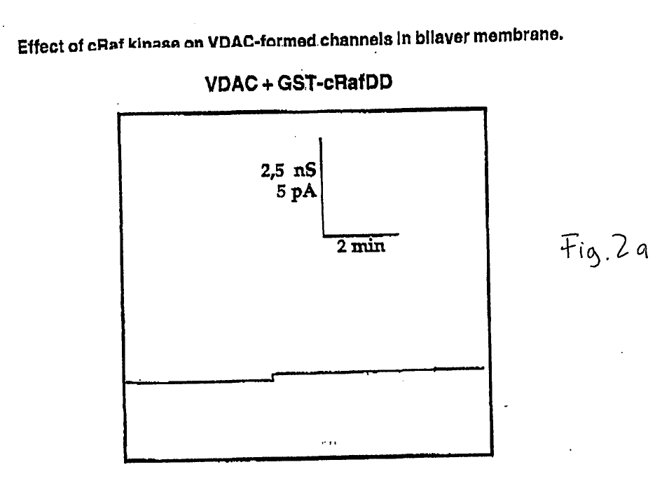

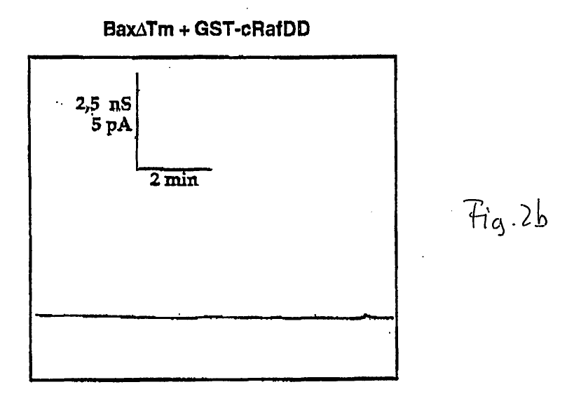

FIG. 2a, b: the permeability of VDAC (a) and BAXΔTM (b) channels after incubation with GST-c-rafYY340/341DD, and

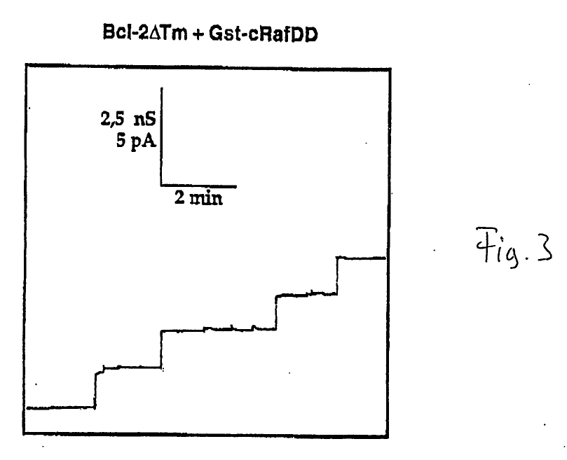

FIG. 3: the permeability of BCL2ΔTM channels after incubation with GST-c-rafYY340/341DD.

### SEQUENCE INFORMATION

The sequence of BAX is known and ready to be called for under the accession number L22473 from www.ncbi.nlm.nih.gov. The sequence of BCL-2 is known and ready to be called for under the accession number M13995 from www.ncbi.nlm.nih. gov. The sequence of VDAC is known and ready to be called for under the accession number NM003374 from www.ncbi.nlm.nih.gov. The sequence of c-raf is known and ready to be called for under the accession number X03484 from www.ncbi. nlm.nih.gov. The sequence of A-raf is known and ready to be called for under the accession number X04790 from www.ncbi.nlm.nih.gov. The sequence of B-raf is known and ready to be called for under the accession number M95712 from www.ncbi.nlm.nih.gov.

### Example 1

### Preparation of Plasmids

BAX and BCL-2 cDNAs are used which code for proteins where the COOH-terminal transmembrane domain is lacking (ATM). In the case of BAX this is BAXAC19, i.e. the amino acids 172-191 are lacking. In the case of BCL-2 this is BCL-2ΔC21, i.e. the amino acids 219-239 are lacking. cDNA coding for BAXΔTM was cut out by digestion with EcoRI/XhoI from the vector pJG4-5 and subcloned into the EcoRI/XhoI sites of pGEX-4T1. cDNA coding for BCL-2ΔTM was expressed in the pGEX-4T1 plasmid (obtained from J. Reed). GST-c-rafYY340/341DD and GST-c-rafL375W were cloned in pFastBac baculoviruses for the expression in Sf9 insect cells.

### Example 2

### Expression and Purification of the Recombinant Proteins

Escherichia coli BL21 DE3 (pLysS) were transformed with BAXΔTM or BCL-2ΔTM constructs and bred on over night at 37° C. in presence of 0.1 mg/ml ampicilin. A single colony of every vector was cultivated in 2×Y-TG medium containing 0.1 mg/ml ampicilin at 37° C. to give an OD (600 nm) value of 0.6. Protein expression was induced by means of 0.1 mM isopropyl-β-D-thiogalactopyranoside for 16 h at 25° C. The cells were obtained by means of centrifugation at 4,000 rpm for 30 min at 4° C. and resuspended in lysis buffer (Tris 50 mM pH 8, NaCl 100 mM, EDTA 1 mM, benzamidine 1 mM, phenylmethylsulfonyl fluoride 1 mM, leupeptin 10 μg/ml, aprotinin 10 μg/ml, β-mercaptoethanol 10 mM). Homogenates of the cells were frozen in liquid nitrogen in presence of 1 mg lysozyme and thawed again prior to ultrasonic treatment. After centrifugation at 28,000 g at 4° C. and for 30 min, the supernatant was incubated with glutathione sepharose beads (Pharmacia Amersham) for 2 h at 4° C. The beads were washed with lysis buffer containing Triton-X100 (0.1%) and NaCl (20 mM) and incubated over night in a buffer (Tris 50 mM pH 8, β-mercaptoethanol 20 mM) containing thrombin (Sigma). Cleaved BAXΔTM and BCL-2ΔTM was further purified on an anion exchange column Resource Q (Pharmacia Amersham), and the proteins were eluted with a NaCl gradient (0-0.5 M).

For obtaining the GST-C-raf proteins, the Sf9 insect cells were infected with the baculoviruses, and cell pellets were frozen and kept at −70° C. The pellets were resuspended in lysis buffer (Tris 25 mM pH 7.6, NaCl 150 mM, Na₄P₂O₇ 10 mM, β-glycerophosphate 25 mM, glycerol 10%, NP-40 0.75%, leupeptin 10 μg/ml, aprotinin 10 μg/ml, benzamidine 1 mM, phenylmethylsulfonyl fluoride 1 mM), homogenized and incubated for 40 min at 4° C. Cell debris was removed by centrifugation at 15,000 g at 4° C. and for 30 min. Supernatants were incubated with glutathione sepharose beads (Pharmacia Amersham) for 2 h at 4° C. The beads were washed the first time with lysis buffer and a second time with lysis buffer containing NaCl (500 mM). GST fusion proteins were eluted with buffer (Tris 50 mM pH 8, aprotinin, leupeptin, benzamidine) containing 20 mM glutathione.

VDAC protein was purified from rat liver under denaturating/renaturating conditions.

### Example 3

### Execution of the Experiments on Lipid Bilayers

The channel-forming proteins were reconstituted in an artificial lipid bilayer. The experimental structure contained a Teflon chamber with two compartments containing aqueous KCl buffers (0.3 M) having a volume of 5 ml, the compartments being connected by a circular hole with a cross section of 0.2 mm². The membrane was generated from a solution of 1% (w/v) diphytanoylphosphateidylcholine (DiphPC, Avanti Polar Lipids, Alabaster, Ala.) in n-decane. The bilayer formation was detectable by that the membrane became optically black with regard to reflected light.

BAXΔTM, BCL-2ΔTM or VDAC protein was added to the KCl buffer in both compartments. The single-channel conductance of the generated pores or channels was measured after application of a constant membrane potential (20 mV) by introduction of one Ag/AgCl electrode each with salt bridges into the buffer of the two compartments. The detected current values were amplified with a current amplifier and recorded as a function of the time.

In order to test the effect of C-Raf kinase on BAXΔTM, BCL-2ΔTM or VDAC channels, the channel-forming proteins (BAXΔTM: 2.4 μg, BCL-2ΔTM: 1.8 μg) were incubated in kinase buffer (Hepes 25 mM pH 7,4, NaCl 150 mM, β-glycerophosphate 25 mM, DTT 1 mM, MgCl₂ 10 mM) in presence of ATP (0.1 mM) with GST-c-rafYY340/341DD (active form of c-raf, GST-c-rafK375W (inactive form of c-raf) or GST alone for 30 min at 30° C. Samples were applied on both sides of the DiphPC membrane in KCl buffer of the compartments, and the single-channel formation was measured. By control measurements it was found that GST-c-RafYY340/341DD, GST-c-rafK375W or GST, each alone, did not form channels in the artificial bilayer membrane.

### Example 4

### Results

In FIG. 1 a, b are shown the results of the permeability measurements with an experimental set as in example 3, VDAC/GST-c-rafL375W (a) and BAXΔTM/GST-c-raf375W (b). It can be seen that channels are formed, i.e. there is no inhibition.

In contrast thereto, it can be taken from FIGS. 2 a, b that the use of the active forms of c-raf, GST-c-rafXX340/341DD in lieu of the inactive form according to FIG. 1 a, b leads to a considerable inhibition of the channel form ation. This is based on the interaction according to the invention of the active c-raf protein with VDAC or BAXΔTM.

Using the active form of c-raf, further a potential influence on BCL-2ΔTM was tested. These results are shown in FIG. 3. It can be seen that obviously there is a channel formation, i.e. c-raf interacts with BCL-2 at any case in a manner not inhibiting the channel formation.

1. An isolated nucleic acid that encodes at least one partial sequence of a protein kinase of the mitogenic signaling cascade, wherein the partial sequence interacts with a porin channel or a nucleic acids capable of hybridizing with said nucleic acid or homologues or derivatives of said nucleic acid.

2. An isolated nucleic acid according to claim 1, wherein the interaction is a reduction in the permeability of porin channels comprising, VDAC, BAX or bacterial porin channels.

3. An isolated nucleic acid according to claim 1, wherein the nucleic acid codes for a protein or peptide containing the sequence c-raf-1 (338 to 627) or an active fragment thereof or a nucleic acid that hybridizes such a nucleic acid.

4. A cDNA prepared from a nucleic acid according to one of claims 1 to 3.

5. An isolated recombinant vector containing a nucleic acid according to one of claims 1 to 3.

6. A protein or peptide encoded by a nucleic acid according to one of claims 1 to 3.

7. A method for treating AIDS, neurodegenerative diseases, for protecting non-transformed cells in a tissue containing cancer cells, or treating bacterial infections comprising administering a pharmaceutical composition comprising a nucleic acid or a protein or peptide encoded by a nucleic acid according to one of claims 1 to 3.

8. A screening method for determining a substance causing the closure of VDAC or BAX or bacterial channels comprising administering a pharmaceutical composition comprising a nucleic acid according to one of claims 1 to 3 or of a protein or peptide coded for hereby.

9. A method for screening for substances capable of modulating porin channels comprising, VDAC, of BAX, or bacterial porin channels, comprising the following steps:
a) providing a solution comprising a porin further comprising, VDAC, BAX, or a bacterial porin that is incubated with a partial sequence of raf,
b) contacting the solution of step a) with a membrane positioned in an electrolyte,
c) applying an electrical potential difference between different sides of the membrane, at time t=0 and beginning with t=0 or at a defined time after t=0, measuring an electrical current caused by the potential difference or a charge transport through the membrane as a function of time,
d) comparing a value measured in step c) to a value which has been measured with an inactive form or an active form of raf.

10. A method for identifying raf activating substances for preparing a pharmaceutical composition for treating a disease according to claim 7, comprising the following steps:
a) reacting target cells with a prospective active ingredient or a mixture of prospective active ingredients, and cultivating the target cells,
b) measuring the expression and/or concentration of raf protein after a pre-defined time, and the obtained measured value is compared to a measured value which has been obtained without addition of a prospective active ingredient, and
c) selecting a prospective active ingredient or a mixture of prospective active ingredients for which an increase of in the expression and/or concentration of raf protein was found in step b).

11. A method for identifying raf activating substances for preparing a pharmaceutical composition for treating a disease according claim 7, comprising the following steps:
a) identifying raf inhibitors naturally occurring in target cells comprising substances that bind to the kinase domain of raf or substances that down-regulate the expression of raf,
b) reacting a substance identified in step a) in a binding assay with a prospective active ingredient or a mixture of prospective active ingredients, and examining the binding capability of the substance with the prospective active ingredient, and
c) selecting a prospective active ingredient or a mixture of prospective active ingredients for which a binding event has been detected in step b).

12. The method of claim 9, wherein the porin comprises a defined partial sequence of VDAC, BAX, or a bacterial porin.

13. The method of claim 10, wherein the raf comprises c-raf-1.

14. The method of claim 10, wherein in step c), a mixture of prospective active ingredients is selected and a deconvolution is performed comprising individual measurement of the active ingredients of the mixture.

15. The method of claim 11, wherein the substance identified in step a) is isolated or purified.

16. The method of claim 11, wherein in step c), a mixture of prospective active ingredients is selected and a deconvolution is performed comprising individual measurement of the active ingredients of the mixture.

17. The method of claim 11, wherein the method further comprises the steps according to claim 10.

18. An isolated nucleic acid according to claim 3, wherein the interaction is a reduction in the permeability of the porin channels comprising VDAC, BAX or bacterial porin channels.
